(19) United States
(12) Patent Application Publication
(10) Pub. No.: US 2018/0008308 A1
(43) Pub. Date: Jan. 11, 2018

(54) VASCULAR ACCESS CONFIGURATION

(71) Applicant: Arstasis, Inc., Redwood City, CA (US)

(72) Inventors: Brian Andrew Ellingwood, Sunnyvale, CA (US); D. Bruce Modesitt, San Carlos, CA (US)

(73) Assignee: Arstasis, Inc., Redwood City, CA (US)

(21) Appl. No.: 15/710,286

(22) Filed: Sep. 20, 2017

Related U.S. Application Data
(63) Continuation of application No. 13/902,599, filed on May 24, 2013, now abandoned.
(60) Provisional application No. 61/652,112, filed on May 25, 2012.

(51) Int. Cl. A61B 17/34; A61M 29/00; A61M 25/06

(52) U.S. Cl. CPC A61B 17/3415; A61M 29/00; A61M 25/0662; A61M 2025/0681

(57) ABSTRACT

One embodiment is directed to a geometric adaptor apparatus for assisting with translumenal vascular access, comprising a dilator adaptor member having proximal and distal ends and defining a dilator adaptor lumen therethrough, wherein the dilator adaptor lumen is defined by an inner diameter profile sized to accommodate insertion of one or more portions of a guidewire, and wherein the dilator adaptor is further defined by an outer diameter profile sized to accommodate at least partial insertion of the proximal end of the dilator adaptor into a dilator member lumen formed through a dilator member, the dilator member being coupleable to an introducer catheter member through an introducer member lumen formed through the introducer member.

### RELATED APPLICATION DATA

The present application is continuation of U.S. patent application Ser. No. 13/902,599, filed on May 24, 2013, which claims the benefit under 35 U.S.C. §119 to U.S. Provisional Application Ser. No. 61/652,112, filed May 25, 2012. The foregoing applications are hereby incorporated by reference into the present application in their entirety.

### FIELD OF THE INVENTION

The present invention relates generally to vascular access systems and techniques, and more particularly to configurations for providing and/or facilitating elongate instrument access across a vascular wall with minimal disruption to surrounding tissue structures.

### BACKGROUND

A number of diagnostic and interventional vascular procedures are now performed translumenally, where an elongate instrument such as a catheter is introduced to the vascular system at a convenient access location—such as the femoral, brachial, or subclavian arteries—and guided through the vascular system to a target location to perform therapy or diagnosis. When vascular access is no longer required, the catheter and other vascular access devices must be removed from the vascular entrance and bleeding at the puncture site must be stopped. One common approach for providing hemostasis is to apply external force near and upstream from the puncture site, typically by manual compression. This method is time-consuming, frequently requiring one-half hour or more of compression before hemostasis. This procedure is uncomfortable for the patient and frequently requires administering analgesics. Excessive pressure can also present the risk of total occlusion of the blood vessel, resulting in ischemia and/or thrombosis. After hemostasis is achieved by manual compression, the patient is required to remain recumbent for six to eighteen hours under observation to assure continued hemostasis. During this time bleeding from the vascular access wound can restart, potentially resulting in major complications. These complications may require blood transfusion and/or surgical intervention.

Bioabsorbable fasteners have also been used to stop bleeding. Generally, these approaches rely on the placement of a thrombogenic and bioabsorbable material, such as collagen, at the superficial arterial wall over the puncture site. This method generally presents difficulty locating the interface of the overlying tissue and the adventitial surface of the blood vessel. Implanting the fastener too far from the desired location can result in failure to provide hemostasis. If, however, the fastener intrudes into the vascular lumen, thrombus can form on the fastener. Thrombus can embolize downstream and/or block normal blood flow at the thrombus site. Implanted fasteners can also cause infection and auto-immune reactions/rejections of the implant.

Suturing methods also have used to provide hemostasis after vascular access. The suture-applying device is introduced through the tissue tract with a distal end of the device located at the vascular puncture. Needles in the device draw suture through the blood vessel wall on opposite sides of the punctures, and the suture is secured directly over the adventitial surface of the blood vessel wall to close the vascular access wound. Generally, to be successful, suturing methods need to be performed with a precise control. The needles need to be properly directed through the blood vessel wall so that the suture is well anchored in tissue to provide for tight closure. Suturing methods also require additional steps for the physician.

In view of the deficiencies of the above methods and devices, a new generation of “self-sealing” closure devices and methods has been developed to avoid the need for implantation of a prosthesis member, and also to minimize the steps and time required for closure of the vascular site. Such self-sealing configurations are available, for example, from Arstasis, Inc., of Redwood City, Calif. under the tradename Axera™, and are described in publications such as U.S. Pat. Nos. 8,083,767, 8,012,168, 8,002,794, 8,002,793, 8,002,792, 8,002,791, 7,998,169, and 7,678,133, each of which is incorporated by reference herein in its entirety.

With self-sealing and other configurations of closure devices, it may be desirable to achieve vascular access with relatively small instruments before dilation up to larger working lumens for subsequent diagnostic or interventional steps. For example, rather than starting with a Seldinger access technique wherein a needle and guidewire set configured to place a conventional 0.035″ diameter guidewire are utilized, a self-sealing access technique may be employed to place a much smaller guidewire, such as an 0.018″ diameter guidewire. With a relatively small guidewire, such as an 0.018″ diameter guidewire, in place by the Seldinger technique, a subsequent process step may be to install an introducer catheter assembly, generally comprising an introducer catheter defining an introducer lumen, and a dilator member configured to fit with in the introducer lumen. The dilator member generally will define its own dilator member lumen through which the guidewire may be threaded, to facilitate an “over-the-wire” installation of the distal portions of the introducer catheter and dilator member into the vascular lumen.

One of the challenges with an over-the-wire installation of a conventional introducer-dilator assembly over a relatively small guidewire, such as an 0.018″ diameter guidewire, is that many readily available off-the-shelf introducer-dilator sets are configured to fit more conventional guidewire diameters through the dilator member lumen, such as diameters in the range of 0.035 inches. The geometric mismatch between a 0.018″ diameter guidewire and a distal end of a dilator member sized for a 0.035″ diameter guidewire, for example, can result in what may be termed an “annular gap” that may form a mechanical edge at the interface between these structures, and insertion of this gap or edge relative to the vascular tissue to place the dilator member and associated introducer catheter distal tips within the vascular lumen may result in unwanted localized tissue trauma, heightened insertion forces, and undesirable localized stress concentrations on portions of the guidewire, dilator member, and/or introducer catheter. There is a need to address this challenge so that conventionally-sized dilator-introducer assemblies, such as those designed for 0.035″ diameter guidewires, may be more optimally utilized with relatively small guidewires, such as those having diameters in the range of 0.018 inches, which may be desirable with procedures such as self-sealing vascular access and closure procedures.

### SUMMARY

One embodiment is directed to a geometric adaptor apparatus for assisting with translumenal vascular access, comprising a dilator adaptor member having proximal and distal ends and defining a dilator adaptor lumen therethrough, wherein the dilator adaptor lumen is defined by an inner diameter profile sized to accommodate insertion of one or more portions of a guidewire, and wherein the dilator adaptor is further defined by an outer diameter profile sized to accommodate at least partial insertion of the proximal end of the dilator adaptor into a dilator member lumen formed through a dilator member, the dilator member being coupleable to an introducer catheter member through an introducer member lumen formed through the introducer member. A maximum outer diameter of the guidewire may be substantially smaller than a minimum inner diameter of the dilator member. Without the dilator adaptor interposed between the guidewire and dilator member, an annular gap may be defined at the intersection of the guidewire and a distal end of the dilator member. The maximum outer diameter of the guidewire may be at least about 25% smaller than the minimum inner diameter of the dilator member. The maximum outer diameter of the guidewire may be about 0.018 inches. The minimum inner diameter of the dilator member may be between about 0.035 inches and about 0.040 inches. The dilator adaptor inner and outer diameter profiles may be configured to substantially make up the difference in fit between the guidewire and dilator member. The dilator adaptor may have a minimum inner diameter of about 0.018 inches, and a maximum outer diameter of about 0.050 inches. The distal end of the dilator adaptor may have a tapered geometry with an outer diameter minimum at its distal tip. At least a portion of the dilator adaptor may have a proximally tapered geometry with an outer diameter minimum located adjacent its proximal end. A friction fit may be formed between the proximally tapered geometry of the dilator adaptor and the dilator member lumen of the dilator member when loading the dilator adaptor into the dilator member lumen. The proximally tapered geometry may be selected such that one size of dilator adaptor can form a friction fit with a range of dilator member lumen geometries. The dilator adaptor, when viewed from distal end to proximal end, may comprise a distal section with a substantially constant outer diameter for a distal section length, tapering up to a midsection with a substantially constant outer diameter for a midsection length, tapering down to a proximal section with a substantially constant outer diameter for a proximal section length, ending in the proximal end. The substantially constant outer diameter of the proximal section may be greater than that of the distal section and less than that of the midsection. Each of the distal section, midsection, and proximal sections may have a substantially homogeneous inner diameter defining the dilator adaptor lumen. The maximum outer diameter of the guidewire may be at least about 0.01 inches smaller than the minimum inner diameter of the dilator member. The dilator adaptor may comprise a polymer selected from the group consisting of: polyethylene terepthalate, polyethylene, high density polyethylene, polypropylene, polytetrafluoroethylene, expanded polytetrafluoroethylene, poly (ethylene-co-vinyl acetate), poly(butyl methacrylate), and co-polymers thereof.

### BRIEF DESCRIPTION OF THE DRAWINGS

FIG. 1A illustrates a geometric misfit scenario wherein a relatively small guidewire is interfaced with a conventionally-sized dilator-introducer assembly.

FIG. 1B illustrates an assembly featuring a dilator adaptor to address a geometric misfit scenario such as that depicted in FIG. 1A.

FIG. 2A illustrates one aspect of a vascular access configuration in accordance with the present invention wherein a relatively small guidewire may be utilized in concert with a conventionally-sized dilator-introducer assembly.

FIG. 2B illustrates one aspect of a vascular access configuration in accordance with the present invention wherein a relatively small guidewire may be utilized in concert with a conventionally-sized dilator-introducer assembly.

FIG. 2C illustrates one aspect of a vascular access configuration in accordance with the present invention wherein a relatively small guidewire may be utilized in concert with a conventionally-sized dilator-introducer assembly.

FIG. 2D illustrates one aspect of a vascular access configuration in accordance with the present invention wherein a relatively small guidewire may be utilized in concert with a conventionally-sized dilator-introducer assembly.

FIG. 2E illustrates one aspect of a vascular access configuration in accordance with the present invention wherein a relatively small guidewire may be utilized in concert with a conventionally-sized dilator-introducer assembly.

FIG. 2F illustrates one aspect of a vascular access configuration in accordance with the present invention wherein a relatively small guidewire may be utilized in concert with a conventionally-sized dilator-introducer assembly.

FIG. 2G illustrates one aspect of a vascular access configuration in accordance with the present invention wherein a relatively small guidewire may be utilized in concert with a conventionally-sized dilator-introducer assembly.

FIG. 2H illustrates one aspect of a vascular access configuration in accordance with the present invention wherein a relatively small guidewire may be utilized in concert with a conventionally-sized dilator-introducer assembly.

FIG. 2I illustrates one aspect of a vascular access configuration in accordance with the present invention wherein a relatively small guidewire may be utilized in concert with a conventionally-sized dilator-introducer assembly.

FIG. 3A illustrates a longitudinal cross sectional view of one embodiment of a dilator adaptor in accordance with the present invention.

FIG. 3B illustrates a longitudinal cross sectional view of another embodiment of a dilator adaptor in accordance with the present invention.

FIG. 4 illustrates a technique conducting a procedure involving a vascular access configuration in accordance with the present invention wherein a relatively small guidewire may be utilized in concert with a conventionally-sized dilator-introducer assembly.

FIG. 5 illustrates a technique conducting a procedure involving a vascular access configuration in accordance with the present invention wherein a relatively small guidewire may be utilized in concert with a conventionally-sized dilator-introducer assembly.

### DETAILED DESCRIPTION

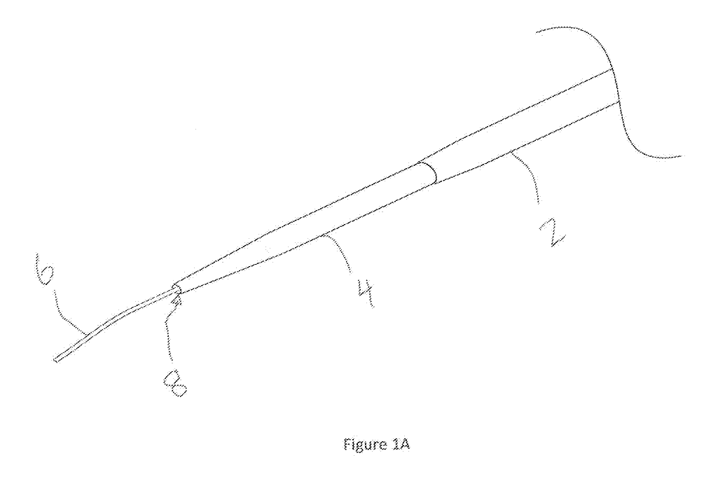

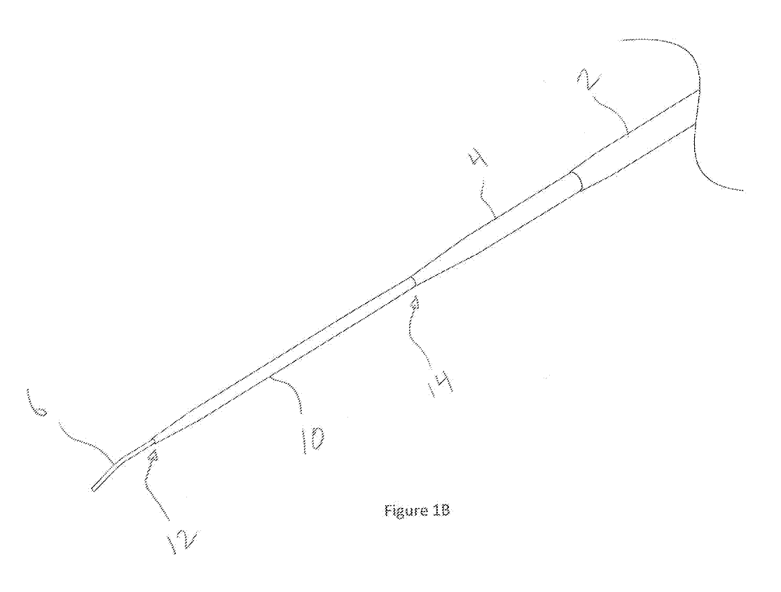

Referring to FIG. 1A, a geometric mismatch scenario is depicted wherein a relatively small guidewire (6), such as a guidewire having a diameter in the range of 0.018 inches, is being utilized in concert with a conventionally-sized dilator-introducer assembly comprising a dilator member (4) coupled through an introducer catheter (2). The dilator member (4) may define a lumen therethrough which has a diameter sized for larger guidewires, in the range of 0.035 inches. As shown in FIG. 1A, and as described above, the geometric mismatch between the guidewire (6) and the inner diameter of the dilator member (4) creates an annular gap (8) or step in geometry, which may unfavorably affect the function of the overall apparatus relative to the nearby pertinent tissue structures. Referring to FIG. 1B, with an appropriate sized and shaped tubular dilator adaptor (10) intercoupled between the dilator member (4) and guidewire (6), the mismatch issue may be substantially, if not completely, mitigated, to produce a desired closely-toleranced fit at the junction (12) between the guidewire (6) and adaptor (10), and at the junction (14) between the adaptor (10) and the dilator member (4).

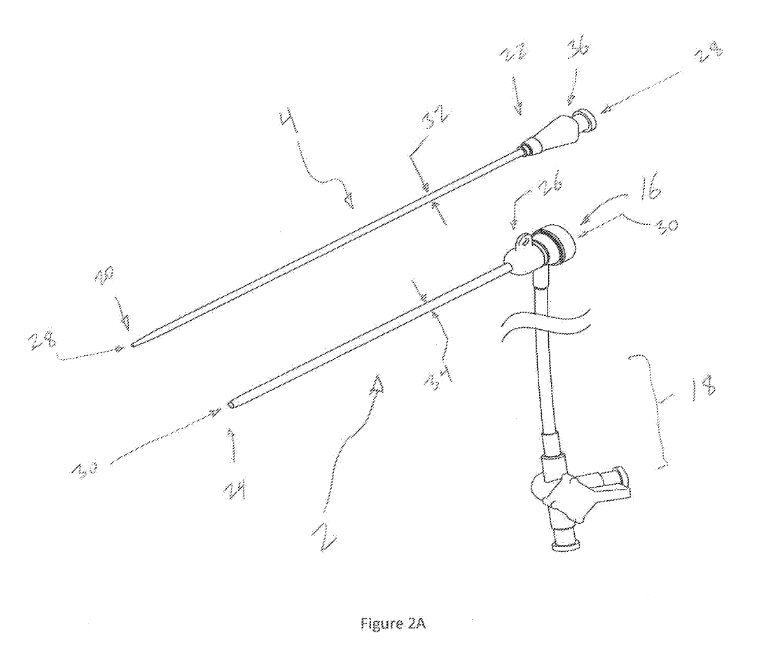

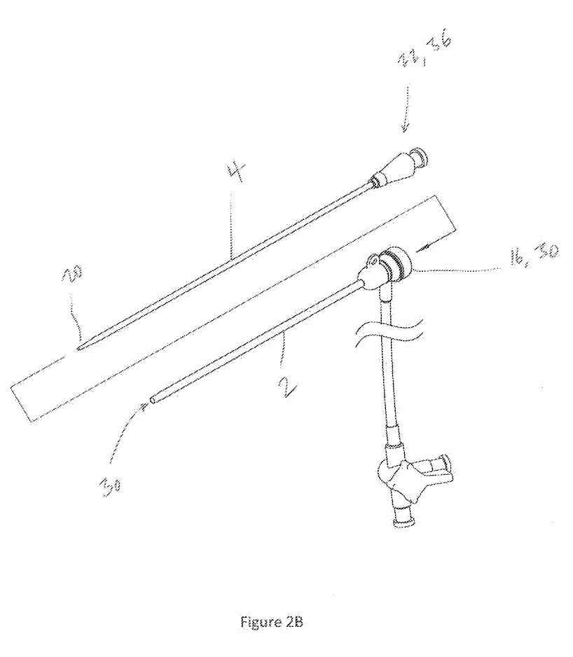

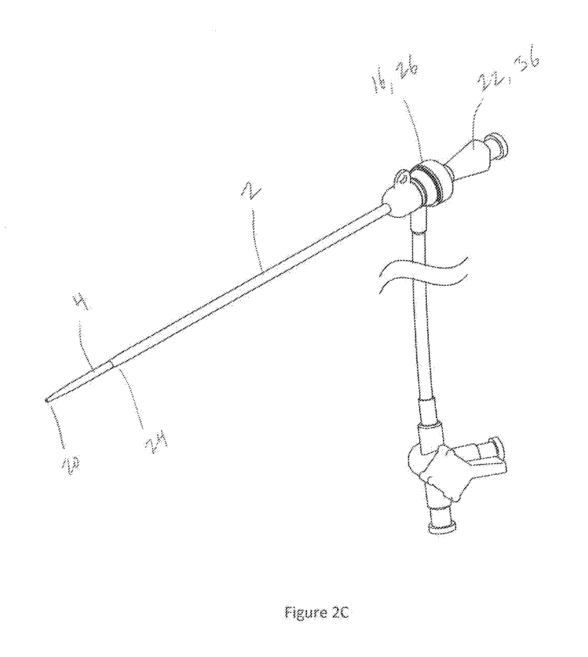

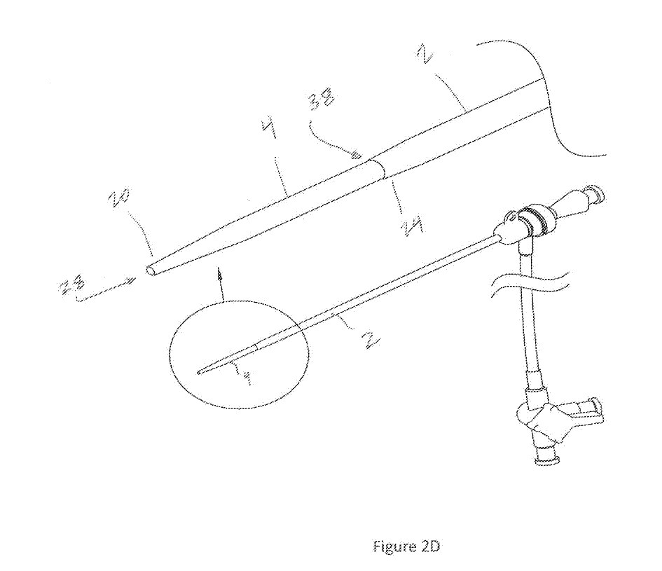

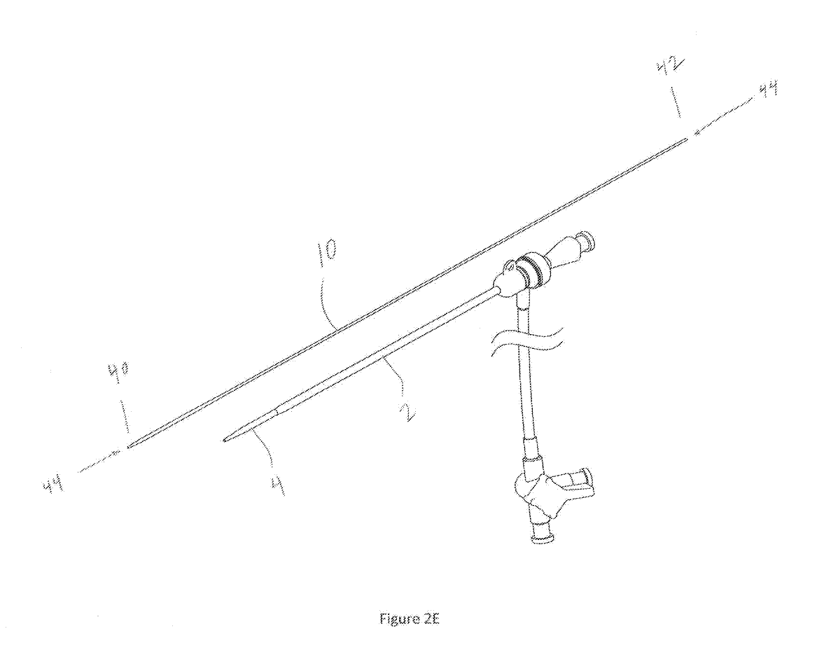

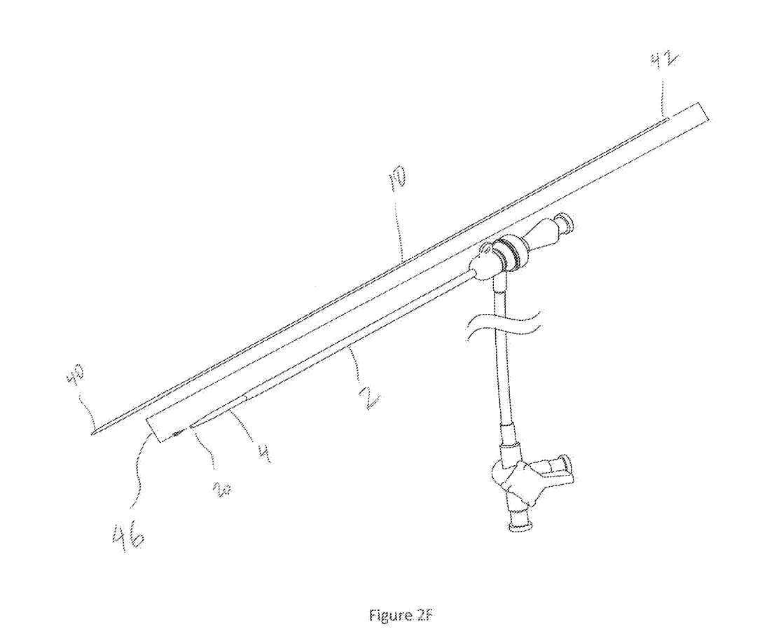

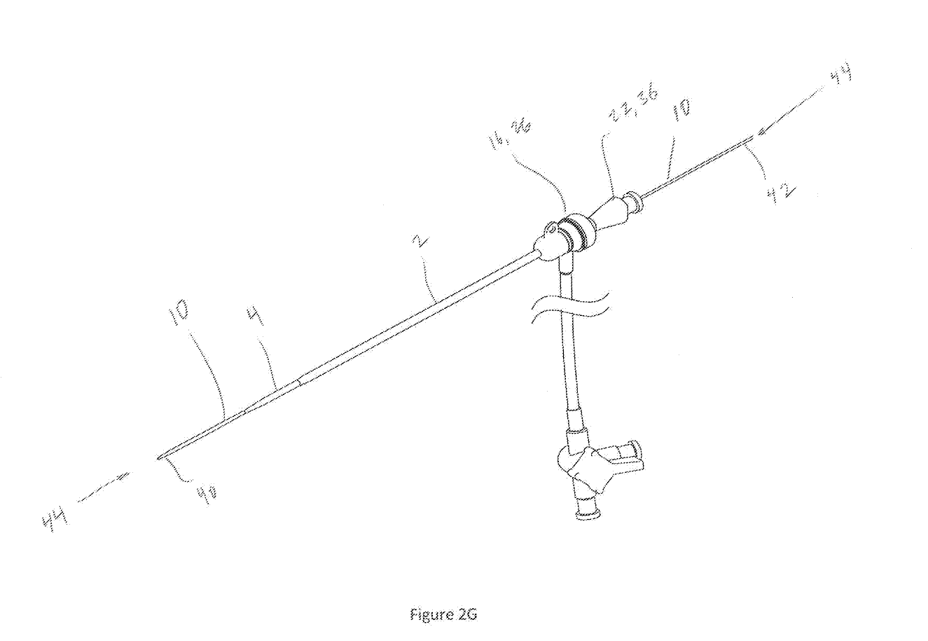

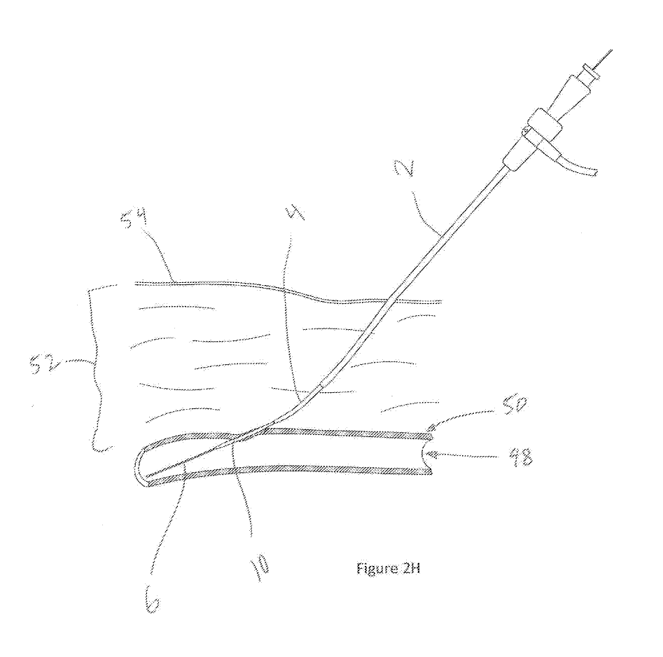

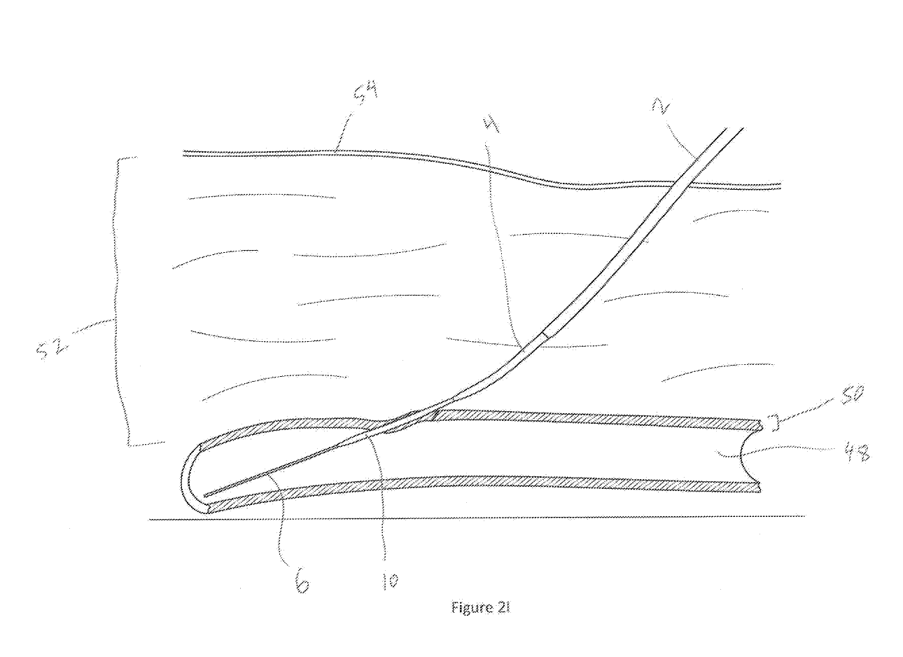

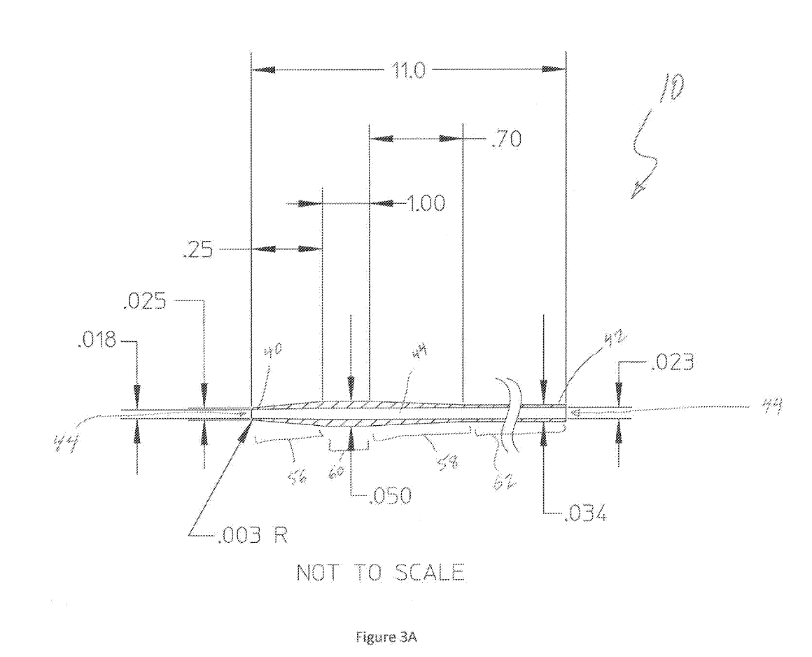

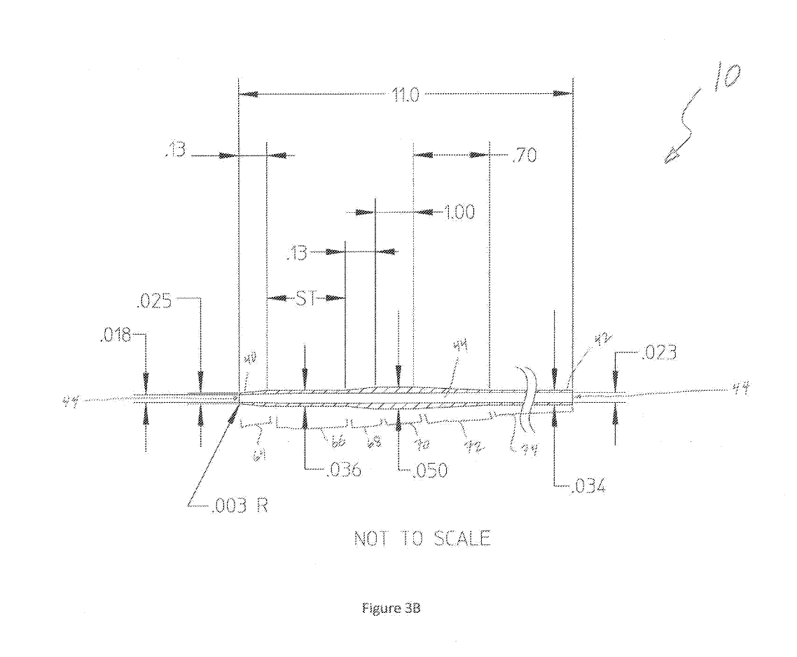

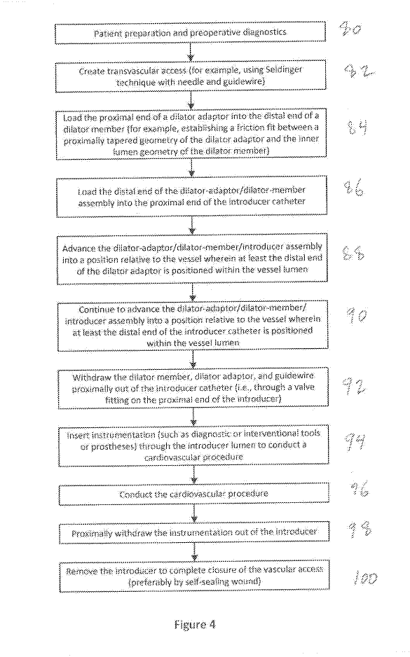

Referring to FIGS. 2A-2I, various aspects of a vascular access system and procedure related thereto are illustrated. FIGS. 3 and 4 illustrate in a flowchart fashion various embodiments of medical procedures involving such vascular access technology.

Referring to FIG. 2A, a conventional dilator member/introducer catheter set is depicted in a disassembled form, comprising a dilator member (4) having proximal (22) and distal (20) ends, a lumen (28) defined therethrough, and a fitting (36) proximally to assist with manipulating and coupling the dilator member (4). The conventional dilator member/introducer catheter set also comprises an introducer catheter or introducer sheath (2) having proximal (26) and distal (24) ends, a lumen (30) therethrough, and a proximal valve assembly (16), to assist with preventing leaks that may otherwise occur through the lumen (30) and around small instruments, such as the dilator member (4) or other diagnostic and/or interventional tools, which may be passed through the lumen (30) and valve (16). Typical dilator member/introducer catheter sets for vascular access, such as those available from providers such as Boston Scientific Corporation, Covidien, Inc., or St. Jude Medical, Inc., are designed to have outer introducer catheter diameters (34) in the range of about 6 French, and have inner dilator member lumen diameters of between about 0.035″ and about 0.038″. The outer diameter (32) of the dilator member (4) typically is configured to be easily slideable through the lumen (30) of the introducer (2), without significant leakage between the two elongate bodies when assembled. Referring to FIG. 2B, the distal end (20) of the dilator (4) may be advanced through the proximal end valve fitting (16) of the introducer catheter (2) and into the lumen (30) of the introducer catheter (2), to form an assembly as shown in FIGS. 2C and 2D, wherein the distal end (20) of the dilator member (4) may be configured to have a tapered geometry and to extend distally past the distal end (24) of the introducer catheter (2), which also may have a tapered distal geometry. Preferably the fit (38) at the interface between the dilator member (4) and introducer catheter (2) is manufactured to be closely toleranced by the manufacturer of the dilator/introducer set, as described above.

Referring to FIG. 2E, in a scenario wherein a relatively small guidewire, such as one in the range of 0.018 inches, is to be utilized in concert with a conventionally-sized dilator/introducer set, such as one having a dilator member lumen diameter of about 0.035 inches, a tubular dilator adaptor member (10) may be added to an assembly to mitigate the geometric mismatch. Generally the dilator adaptor (10) has proximal (42) and distal (40) ends and comprises a small lumen (44) defined therethrough to accommodate passage of a small instrument such as a guidewire. As shown in FIG. 2F, in one embodiment, the dilator adaptor (10) is configured to be inserted proximal end (42) first into the distal end (20) of the dilator member (4), and this assembly may occur before or after the dilator member is assembled into the working lumen of the introducer catheter (2). Referring to FIG. 2G, a resulting assembly is depicted, with the dilator adaptor (10) inserted through the working lumen of the dilator member (4), which is inserted through the working lumen of the introducer catheter (2). A small working lumen (44) is maintained through the dilator adaptor (10) to accommodate passage of a guidewire or other small instrument.

Preferably at least one portion of the proximal end geometry of the dilator adaptor (10) comprises a proximal taper (tapering to smaller outer diameter as one measures incrementally closer to the proximal end of the dilator adaptor) which is configured to interface with the inner lumen geometry of the working lumen of the associated dilator member (4) in such a manner that the dilator adaptor (10) may be pushed up into the distal end of the dilator member (4) until a friction fit is established. Preferably the proximal taper geometry of the dilator adaptor (10) is configured to not only accommodate one guidewire/dilator mismatch scenario (i.e., such as one wherein an 0.018″ outer diameter guidewire is to be utilized with a dilator member having an inner lumen diameter of about 0.035″), but also a substantially broad range of mismatch scenarios (including one wherein an 0.018″ outer diameter guidewire is to be utilized with a dilator member having an inner lumen diameter of about 0.038″, as well as a myriad of other mismatch scenarios which may be greater in mismatch dimensions).

Referring to FIG. 2H, in practice, with a guidewire (6) already installed into a position wherein the distal end of the guidewire extends into a blood vessel lumen (48), and the remainder of the guidewire (10) extends proximally across the vessel wall (50), across other related tissue structures (52), and across the skin (54) of the patient, to extend proximally, generally outside of the patient, an assembly of the dilator adaptor (10), dilator member (4), and introducer catheter (2) may be advanced in an “over-the-wire” technique to place at least a portion of such assembly within the vascular lumen (48). A closer view is presented in FIG. 2I. The assembly may be further advanced until the distal end of the introducer catheter is positioned within the vascular lumen (48), after which the dilator member (4) and dilator adaptor (10) may be withdrawn proximally to make room for other diagnostic and/or interventional tools, such as catheters, imaging devices, and prostheses such as stents which may be passed through the working lumen of the introducer. Subsequently, the tools may with withdrawn, as well as the guidewire and introducer sheath, to complete closure of the trans-vascular access port or wound. As described above, in one embodiment, the trans-vascular access point across the vessel wall (50) may be configured to be a self-sealing access point, which is designed to self-seal after withdrawal of the pertinent instrumentation.

Referring to FIG. 3A, a longitudinal cross sectional view of one embodiment of a dilator adaptor (10) is depicted with dimensions in inches. The most distal portion starting from the distal end (40) may comprise a tapered geometry (56) to ultimately assist with pushing deployment into the pertinent tissue structures. A mid-portion (60) may have a substantially homogeneous outer diameter for a given length. Next a proximally tapered portion (58) may assist with establishing a friction fit with an associated inner lumen geometry of a dilator member, as described above. A most proximal portion (62) up to the proximal end (42) may have a substantially constant outer diameter for a given length.

Referring to FIG. 3B, a longitudinal cross sectional view of another embodiment of a dilator adaptor (10) is depicted with dimensions in inches. The most distal portion starting from the distal end (40) may comprise a tapered geometry (64), followed by a portion (66) having a substantially constant outer diameter for a given length, followed by another tapered portion (68), a midportion (70) which may have a mild taper either proximally or distally or be substantially constant in outer diameter for a given length, followed by a proximally tapered portion (72) which may assist with establishing a friction fit with an associated inner lumen geometry of a dilator member, as described above. A most proximal portion (74) up to the proximal end (42) may have a substantially constant outer diameter for a given length. The dilator adaptor may comprise a polymer selected from the group consisting of: polyethylene terepthalate, polyethylene, high density polyethylene, polypropylene, polytetrafluoroethylene, expanded polytetrafluoroethylene, poly (ethylene-co-vinyl acetate), poly(butyl methacrylate), and co-polymers thereof.

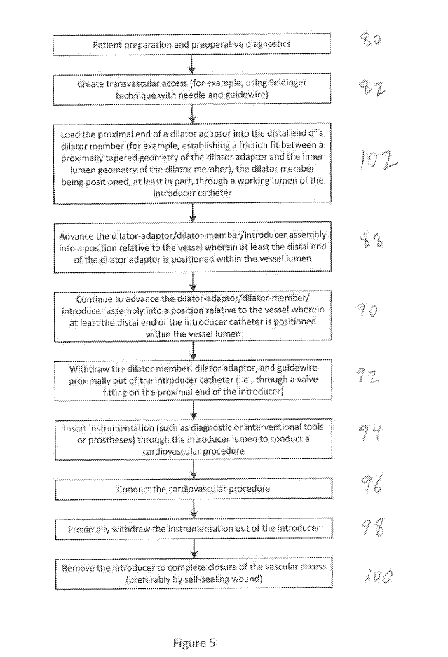

Referring to FIG. 4, after patient preparation and preoperative diagnostics (80), access may be created (for example, by crossing with an access needle and leaving behind a guidewire, as in a Seldinger technique) (82). An operational assembly may be formed which combines the at least a portion of the guidewire through the dilator adaptor lumen, at least a portion of the dilator adaptor through the dilator member lumen, and at least a portion of the dilator member through the introducer catheter lumen. Such an assembly may be accomplished by first assembling the adaptor and dilator member together, then placing this assembly into the introducer for further advancement over the guidewire into the vessel, as shown in the embodiment of FIG. 4 (elements 84, 86); referring to FIG. 5 (102), such a sub-process may comprise combining the adaptor into an already-assembled dilator member-introducer catheter subassembly. The dilator-adaptor/dilator member/introducer assembly may then be advanced in an “over-the-wire” configuration (i.e., with the proximal end of the guidewire (and additional portions thereof following) being advanced into the distal end of the dilator-adaptor and associated dilator member and introducer as this assembly is advanced over the guidewire) into a position relative to the vessel wherein at least the distal end of the dilator adaptor is positioned within the vessel lumen (88). With the distal end of the introducer having access to the vascular lumen (90), the dilator member and dilator adaptor may be withdrawn (92) along with the guidewire, and other instrumentation may be advanced through the working lumen of the introducer catheter or sheath (94) to conduct a procedure (96), after which the instrumentation may be withdrawn out of the introducer (98), and the remaining introducer may be withdrawn to complete the closure, which preferably has been set up to be a self-sealing closure (100).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A geometric adaptor apparatus for assisting with translumenal vascular access, comprising:
a dilator adaptor member having proximal and distal ends and defining a dilator adaptor lumen therethrough, wherein the dilator adaptor lumen is defined by an inner diameter profile sized to accommodate insertion of one or more portions of a guidewire, and wherein the dilator adaptor is further defined by an outer diameter profile sized to accommodate at least partial insertion of the proximal end of the dilator adaptor into a dilator member lumen formed through a dilator member, the dilator member being coupleable to an introducer catheter member through an introducer member lumen formed through the introducer member.

2. The apparatus of claim 1, wherein a maximum outer diameter of the guidewire is substantially smaller than a minimum inner diameter of the dilator member.

3. The apparatus of claim 2, wherein without the dilator adaptor interposed between the guidewire and dilator member, an annular gap would be defined at the intersection of the guidewire and a distal end of the dilator member.

4. The apparatus of claim 2, wherein the maximum outer diameter of the guidewire is at least about 25% smaller than the minimum inner diameter of the dilator member.

5. The apparatus of claim 2, wherein the maximum outer diameter of the guidewire is about 0.018 inches.

6. The apparatus of claim 5, wherein the minimum inner diameter of the dilator member is between about 0.035 inches and about 0.040 inches.

7. The apparatus of claim 3, wherein the dilator adaptor inner and outer diameter profiles are configured to substantially make up the difference in fit between the guidewire and dilator member.

8. The apparatus of claim 6, wherein the dilator adaptor has a minimum inner diameter of about 0.018 inches, and a maximum outer diameter of about 0.050 inches.

9. The apparatus of claim 1, wherein the distal end of the dilator adaptor has a tapered geometry with an outer diameter minimum at its distal tip.

10. The apparatus of claim 1, wherein at least a portion of the dilator adaptor has a proximally tapered geometry with an outer diameter minimum located adjacent its proximal end.

11. The apparatus of claim 10, wherein a friction fit may be formed between the proximally tapered geometry of the dilator adaptor and the dilator member lumen of the dilator member when loading the dilator adaptor into the dilator member lumen.

12. The apparatus of claim 11, wherein the proximally tapered geometry is selected such that one size of dilator adaptor can form a friction fit with a range of dilator member lumen geometries.

13. The apparatus of claim 1, wherein the dilator adaptor, when viewed from distal end to proximal end, comprises a distal section with a substantially constant outer diameter for a distal section length, tapering up to a midsection with a substantially constant outer diameter for a midsection length, tapering down to a proximal section with a substantially constant outer diameter for a proximal section length, ending in the proximal end.

14. The apparatus of claim 13, wherein the substantially constant outer diameter of the proximal section is greater than that of the distal section and less than that of the midsection.

15. The apparatus of claim 14, wherein each of the distal section, midsection, and proximal sections has a substantially homogeneous inner diameter defining the dilator adaptor lumen.

16. The apparatus of claim 2, wherein the maximum outer diameter of the guidewire is at least about 0.01 inches smaller than the minimum inner diameter of the dilator member.

17. The apparatus of claim 1, wherein the dilator adaptor comprises a polymer selected from the group consisting of: polyethylene terepthalate, polyethylene, high density polyethylene, polypropylene, polytetrafluoroethylene, expanded polytetrafluoroethylene, poly (ethylene-co-vinyl acetate), poly(butyl methacrylate), and co-polymers thereof.
